(19) United States
(12) Patent Application Publication
(10) Pub. No.: US 2025/0072867 A1
(43) Pub. Date: Mar. 6, 2025

(54) MODULAR ACOUSTIC SENSOR ASSEMBLIES

(71) Applicant: University of Southern California, Los Angeles, CA (US)

(72) Inventors: Qifa Zhou, Hacienda Heights, CA (US); Robert G. Wodnicki, Los Angeles, CA (US); Yizhe Sun, Los Angeles, CA (US)

(21) Appl. No.: 18/819,488

(22) Filed: Aug. 29, 2024

Related U.S. Application Data
(60) Provisional application No. 63/536,056, filed on Aug. 31, 2023.

(51) Int. Cl. A61B 8/00; B33Y 80/00; G01S 7/52; G01S 15/89

(52) U.S. Cl. CPC A61B 8/4494; A61B 8/4411; B33Y 80/00; G01S 7/52082; G01S 15/8925

(57) ABSTRACT

A modular acoustic sensor assembly and method of forming the same is disclosed comprising: a two-dimensional acoustic sensor array; an electrically conducting and acoustically attenuating interposer; an assembly connection layer; and a first routing subassembly, wherein at least one electrical routing plane of the interposer is substantially parallel to a principal electrical routing plane of the routing substrate.

### CROSS-REFERENCE TO RELATED APPLICATIONS

This application claims priority to U.S. Provisional Patent Application No. 63/536,056, entitled “Modular Acoustic Sensor Assemblies”, filed Aug. 31, 2023, which is hereby incorporated by reference in its entirety.

### FIELD

Disclosed herein are acoustic sensor assemblies and methods for making the same and, more particularly, large area two-dimensional modular acoustic sensor assemblies that are configured to operate at high frequency and fine pitch for imaging, e.g., macro-scalar biological tissues at the micro-level.

### BACKGROUND

Ultrasound is a highly versatile imaging modality with wide clinical use for imaging of macro-scalar anatomy including liver, kidney, breast, and heart. Axial and lateral resolution in ultrasound is determined by the wavelength of the imaging system with clinical imaging occurring in the range of 1-10 MHz for an axial resolution of between 1.5 mm and 150 μm. Systems for ophthalmology and small animal imaging in vivo operate in the range of 10-50 MHz (150 μm to 30 μm resolution), and imaging at the tissue and cellular level can be done using systems operating in the range of 50-200 MHz (30 μm to 7.5 μm resolution). Typical ultrasound systems utilizing 1D or single element probes, acquire single cross-sectional image planes parallel to the axial imaging direction. These can be used to build up volumetric images by mechanically translating the imaging scan head or probe. Electronically scanned two-dimensional arrays of ultrasound transducers can be further used to create volumetric image datasets that are acquired at high framerates (10-100 fps) in real-time. Such volume imaging arrays have become more standard in the clinical imaging space in the last 10-15 years for imaging the fetus in the womb and for echocardiographic studies, however, they are currently limited to low-resolution applications (e.g. 0.5 mm and above).

In ultrasound, while the imaging resolution is related to the wavelength of the operating frequency, the pitch between transducer elements in an array is directly related to the wavelength as well. Linear arrays, producing axial image slices typically have λ-pitch element spacing, while 2D arrays, which can produce volumetric datasets, require λ/2-pitch element spacing. Recent advances in electronics miniaturization and packaging and assembly as well as novel ultrasound transducer technologies have made possible the dense integration of fine pitch elements enabling 2D arrays to be fabricated at λ/2-pitch for frequencies above 5 MHz and less than 10 MHz. These have been used for example for intracardiac (ICE) and transesophageal echocardiography (TEE) 3D volumetric imaging in real-time (so-called 4D imaging). While these systems can produce fine images at the macro-scalar level for observation of tissues in vivo they are not yet suitable for imaging at higher frequencies required for cellular level resolution.

There are two principal ways in which the current state of the art has addressed higher frequency and fine resolution volumetric imaging. The first involves building 2D phased arrays at fine pitch using direct assembly of the 2D array to the surface of a custom micro-chip which comprises unit interface cells and assembly pads that are pitch-matched to the pitch of the 2D transducer array. These devices have been limited to pitches above 100 μm due to the fact that the required high voltage CMOS circuitry for ultrasound transmit circuits is large and difficult to shrink below 100 μm pitch. The second method for realizing volumetric imaging at high frequency is to mechanically sweep a production linear array to build up the 3D volume similar to how CT and MRI machines build up 3D volumes at the macro-scalar level. This is a tried-and-true technique in medical imaging; however, it is also extremely slow since it is limited by the time of flight of ultrasound in the tissue as well as the speed of mechanical translation of the linear array ultrasound probe. Therefore, applications of real-time volumetric ultrasound enabled by 2D arrays or swept linear arrays are currently limited to macro-scalar resolution and non-real time imaging speeds.

In particular, there are very significant applications for 2D arrays with real-time volumetric imaging capability at high frequency (10 MHz-50 MHz) which remain unaddressed at this time. These include real-time volumetric small-animal imaging for preclinical drug development studies, ophthalmological applications, imaging of skin for cancer screening, and intravascular ultrasound (IVUS) for evaluation of morphology of vulnerable plaques. In addition, a host of new applications could benefit from widely available high frequency volumetric imaging arrays including rapid imaging of organoids for drug development and personalized medicine, imaging of 3D-printed living tissues for morphological validation of printed structures, and non-contact tissue characterization at the micro-scalar level. All of these applications remain unserved due to the lack of a viable technology for fabrication of 2D arrays at pitches below 100 μm.

Additional traditional means for imaging at the micro-scalar level include micro-CT which is bulky and uses ionizing radiation which can damage living tissues, as well as well-known optical methods. In the prior art utilizing optical imaging modes, there exist a number of imaging techniques and systems for obtaining single plane and volume acquisitions at the macro and micro-scalar levels. These include such non-invasive optical sectioning microscopy methods as confocal or multiphoton laser scanning microscopy as well as light-sheet fluorescence microscopy (LSFM). These methods obtain 3D volumes by scanning a thin axially focused imaging plane through multiple sections of the imaged tissue and thereby reproducing the complete 3D volume. An important distinguishing feature of these systems is that they are fundamentally limited in their ability to produce real-time volumetric images due to the fact that the imaging sections are scanned mechanically. This can be done either by mechanically translating the specimen stage in the axial direction (e.g. for confocal microscopy) or by scanning the laser light sheet in an LSFM type system using a translating mirror. These systems also have a limited axial depth of field which varies inversely with the frame-rate. Imaging larger depth samples requires long acquisition times due to the mechanically translated optical image focal planes. This precludes the option of obtaining high optical resolution for larger structures that are moving in real-time. The use of these systems in high resolution imaging for real-time volumetric imaging at micro-scalar levels with reasonable depth of field is challenging due to the limited penetration of light in tissue as well as the time-consuming mechanical scanning process. In addition, these systems are bulky, expensive, and sensitive to mechanical vibration which precludes their use as widely available and low-cost tools for general imaging applications.

Therefore, there exists a need for volumetric imaging at high frequency and fine resolution producing real-time in-vivo datasets of macro-scalar structures in an efficacious and timely manner.

### SUMMARY

A modular acoustic sensor assembly for operation at high frequency is disclosed comprising: a two-dimensional acoustic sensor array; an electrically conducting and acoustically attenuating interposer; an assembly connection layer; and a first routing subassembly, wherein at least one electrical routing plane of the interposer is substantially parallel to a principal electrical routing plane of the routing subassembly.

A process for building a modular acoustic sensor assembly is disclosed comprising the steps of: micro-machining a piezo-electric single-crystal material to form a 2D array of elements; bonding the micro-machined array to an interposer; and one of: bonding a 3D-printed metal pin and connection link matrix to an edge face of a first routing subassembly using a thermo- compression or solder reflow process; singulating the 3D-printed metal pins from each other by cutting the connection link matrix using a dicing saw or a laser cutting process; and attaching the micro-machined array and the interposer to the first routing subassembly by inserting the 3D-printed metal pins into the interposer channel material; or attaching a 3D-printed metal pin and connection link matrix to the micro-machined array and interposer by inserting the 3D-printed metal pins into channel material of the interposer; singulating the 3D-printed metal pins from each other by cutting the connection link matrix using a dicing saw or a laser cutting process; and bonding the edge face of the first routing subassembly to the 3D-printed metal pins using the thermo-compression or solder reflow process.

A process for building a modular acoustic sensor assembly is disclosed comprising the steps of: micro-machining a piezo-electric single-crystal material to form a 2D array of elements; bonding the micro-machined array to an interposer; bonding a 3D-printed metal pin and connection link matrix to an edge face of a first routing subassembly using a thermo- compression or solder reflow process; singulating the 3D-printed metal pins from each other by cutting the connection link matrix as part of the press-fit assembly process using protruding tabs on the bottom surface of the mating interposer; and attaching the micro-machined array and the interposer to the first routing subassembly by inserting the 3D-printed metal pins into the interposer channel material.

### BRIEF DESCRIPTION OF THE DRAWINGS

These and other features and aspects of modular acoustic sensor assemblies and methods of making the same as disclosed herein will be appreciated as the same becomes better understood by reference to the following detailed description when considered in connection with the accompanying drawings where:

FIG. 1 is a perspective view of an example embodiment as disclosed herein;

FIG. 2 is a perspective view of an example embodiment as disclosed herein;

FIG. 3 is a perspective view of an example embodiment as disclosed herein;

FIG. 4 is a plan view of an example embodiment as disclosed herein;

FIG. 5 is a flow chart of steps used for making an example embodiment as disclosed herein;

FIG. 6 are perspective views of an example embodiment as disclosed herein;

FIG. 7 are perspective views of an example embodiment as disclosed herein;

FIG. 8 is a perspective view of an example embodiment as disclosed herein;

FIG. 9 are perspective and cross-sectional views of an example embodiment as disclosed herein;

FIG. 10 are views of an example embodiment as disclosed herein;

FIG. 11 are views of an example embodiment as disclosed herein;

FIG. 12 are views of an example embodiment as disclosed herein;

FIG. 13 are views of an example embodiment as disclosed herein;

FIG. 14 is a flow chart of steps used for making an example embodiment as disclosed herein;

FIG. 15 is a flow chart of steps used for making an example embodiment as disclosed herein;

FIG. 16 is a flow chart of steps used for making an example embodiment as disclosed herein;

FIG. 17 is a flow chart of steps used for making an example embodiment as disclosed herein;

FIG. 18 is a view of an example embodiment as disclosed herein;

FIG. 19 is a view of an example embodiment as disclosed herein;

FIG. 20 is a perspective view of an example embodiment as disclosed herein;

FIG. 21 is a perspective view of an example embodiment as disclosed herein;

FIG. 22 is a cross-sectional view of the example embodiment as disclosed herein; and

FIG. 23 is a perspective view of an example embodiment as disclosed herein.

### DESCRIPTION

It is desirable to support the efficient imaging of macro-scalar tissue constructs with micro-scalar resolution and provide 3D volume datasets. One important application of the present disclosure is to provide imaging for monitoring of efficacy of drug candidates as part of pre-clinical assessment. In the study of cancer biology and specifically the inhibition of proliferation of cancerous cells by specifically engineered therapeutic agents, the three-dimensional morphology of the tissue growth over time can be an important indicator of drug efficacy. One way to track the growth of these tissues over time for in-vivo animal models is using high resolution ultrasound scanning. For in vitro analysis of tissue organoids, it would be advantageous to be able to use ultrasound scanning for tissue tracking in large cohort in vitro studies. The present disclosure could also be useful for monitoring biological tissue constructs that are realized using 3D printing, for example, to create organs from living cells. The present disclosure could be used to monitor the construction of these tissue constructs throughout the printing process to assess the efficacy of the process and evaluate the evolution of important biological and structural parameters throughout the printing process.

To realize an ultrasound array to address the goals of the present disclosure, it is required that the surface of the array be large and with fine pitch for high resolution and high frequency operation These goals are advantageously addressed as described here below. In particular, one challenge that has not been adequately addressed to date in the prior art is that standard assembly methods rely on vertical flex integration which is problematic due to the vias in standard electronic substrates being 50 μm or greater in width. Large wide 2D and 1.75D arrays are needed to improve azimuthal image quality but difficult to implement with standard 2D array processes due to this limitation in via width. Another way to implement large arrays that does not require vias is by direct integration of the 2D array elements to the surface of an Application Specific Integrated Circuit (ASIC). However, this is challenging due to the fact that all the respective electronics in such pitch-matched element architectures are limited to the small size of the array elements which leads to greater expense, reduced functionality and performance and low yield of large ASIC die. One way to alleviate this bottleneck is by separating the ASICs from the transducer array to free up the electronics for higher quality implementation with less trade-offs. This also makes it possible to realize modular architectures where it is possible to implement a single ASIC design across multiple array types rather than having different ASICs designed for each special case of array architecture which is both time-consuming and expensive.

A critical issue with previously proposed 2D array implementations at high frequency is the requirement to dice kerfs through the acoustic stack down through the intervening backing layer to the routing substrate due to the requirement for the use of conducting epoxy for assembly. The use of conducting epoxy at low frequencies is advantageous due to the fact that it is not sensitive to variation in coplanarity in the transducer/backing acoustic stack and the mating electronics routing substrate. The conducting epoxy essentially fills in the gaps in the surface variation and provides uniform connection of the two sensor and electronics layers with high yield. However, the process for this assembly requires the continuous conducting epoxy layer to be singulated using mechanical or laser dicing means in order to electrically isolate each of the transmit/receive channels from their neighboring connections At low frequencies, where the pitch between elements is large (e.g., >100 μm) this singulation process can be accomplished using standard dicing methods where the minimal blade width yields a kerf of around 20 μm. However, once the pitch between elements is decreased below 100 μm, the fractional area in each element that is taken up by the dicing kerf begins to grow to an unacceptable size. A further limitation is that the depth of cutting for very fine blades is limited. This is problematic because the array backing needs to typically be diced through as well, and therefore the thickness of this backing is reduced to accommodate the limited blade depth. Since the efficiency of the acoustic attenuation function of the backing is directly related to its thickness, this coupling of the trade-off can significantly compromise the acoustic performance of the array elements leading to reduced axial resolution due to much longer acoustic ringdown. For this reason, the use of these dicing methods for element singulation patterning is unacceptable for high frequency 2D arrays. Therefore, there exists a need for an assembly method at very fine pitch accommodating surface planarity variations on the array and electronics substrates that maintains a high acoustic area free of dicing kerf requirements.

One way to realize electrical interconnection of arrays of elements to a respective routing substrate with high yield is by providing collapsing assembly points. This is the standard methodology used in solder reflow for attaching large signal count electronic packages (e.g., micro-BGA) to printed circuits using reflow of solder balls which deform to accommodate the differences in coplanarity between chip and substrate. Solder reflow is not amenable to standard acoustic arrays where low temperature resin-based materials are used extensively for mechanical construction and acoustic isolation and matching. Therefore, a low temperature (<70C) assembly method is required, preferably with room temperature curing.

In the present disclosure, these issues are addressed by providing arrays of assembly points on both an acoustically active electrical interposer backing and on the surface of the mating electrical routing assembly. The assembly points are constructed to collapse into each other so as to regularize the difference in surface planarity between the two components of the assembly. In addition, precise assembly stops are provided to ensure that the connection points collapse into each other in a controlled manner with a specific precise depth. These assembly stops further provide a large surface area for mechanically bonding the two mating components to each other thereby improving reliability of the construction. In addition, separating the acoustic stack from the electronics routing allows for these two different components to be independently optimized which improves yield and performance and reduces cost. In essence, the realized structure can be thought of as a connector that is used to attach different components to each other with all of the advantages inherent in such an arrangement.

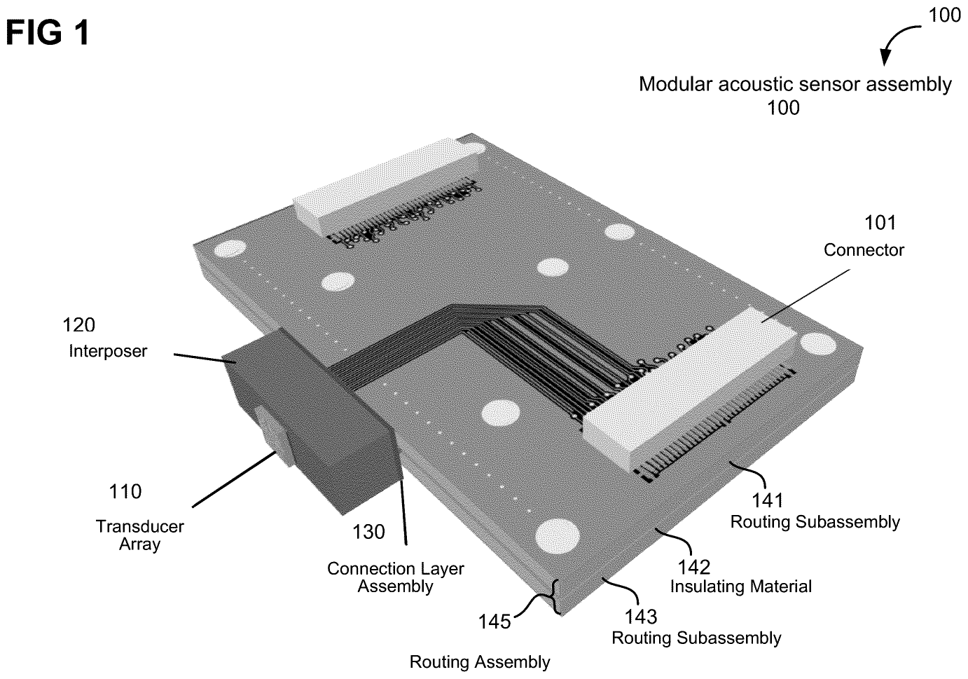

In a first embodiment illustrated in FIG. 1, the present disclosure describes a modular acoustic sensor assembly 100 for operation at high frequency comprising: a two-dimensional acoustic sensor array 110, an electrically conducting and acoustically attenuating interposer 120, an assembly connection layer 130, and a routing subassembly 141 wherein at least one electrical routing plane of the interposer 120 is substantially parallel to the principal electrical routing plane of the routing subassembly 141. The sensor array 110 may comprise transducer elements. The transducer elements may comprise a pitch in the range of 150 μm down to as small as 5 μm depending on the operating frequency of the array

In an example embodiment, the interposer 120 may comprise a grid of tubes embedded in a solid block of material. These tubes are filled using a conducting epoxy which is also acoustically attenuating. The structure thereby serves both as an interconnect and acoustic backing. This structure may be created by 3D printing using a micro-resolution capable acrylic printer to print the block of material with tubes in it or by injection molding.

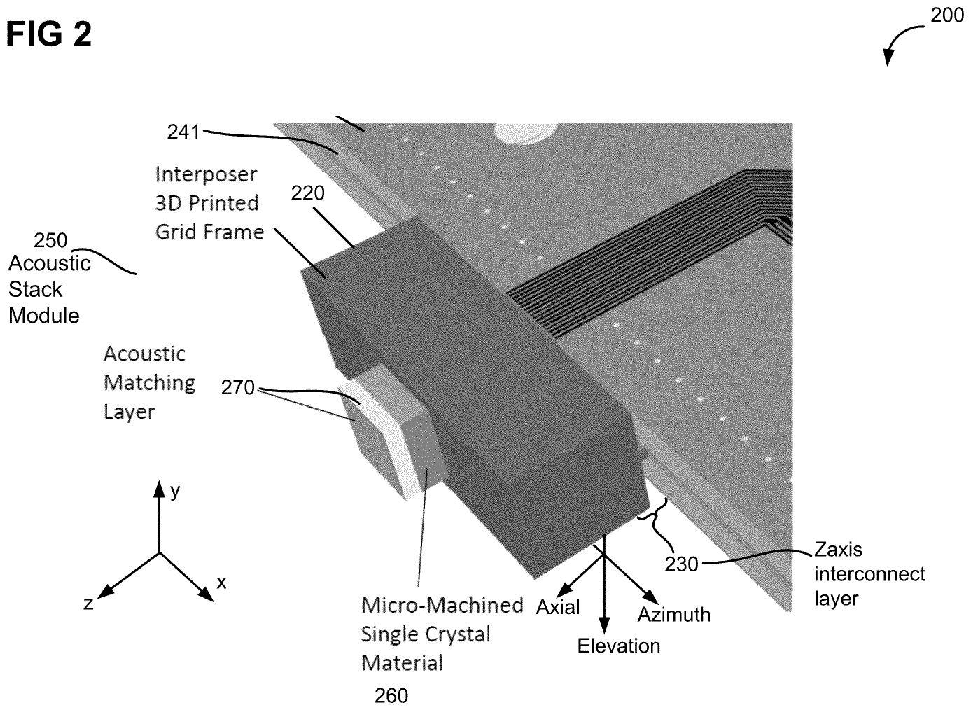

In an example embodiment, an acoustic stack module 250 is illustrated in FIG. 2. The acoustic stack module 250 may comprise the interposer 220 with the 2D array, an acoustic piezo layer 260 and acoustic matching layers 270. In this example embodiment, the interposer 120 may comprise an acrylic grid and conductive epoxy pillars. The acoustic piezo layer 260 may be assembled to the interposer 220 using an Isotropic Conducting Epoxy, with elements being defined by mechanical or laser dicing, or isotropic etching of kerfs between elements. The acoustic piezo layer 260 may also be assembled using a non-conducting resin epoxy in which case further dicing to define the singulated elements is not required. In an example embodiment, the acoustic piezo layer 260 is a micro-machined single crystal material. This example embodiment is advantageous due to the fact that element pitch is currently limited by the minimum attainable width (20μm) and depth (300 μm) of mechanically diced epoxy kerfs. The acoustic piezo layer 260 may also be assembled to the interposer 220 using an Anisotropic Conducting Epoxy with elements being pad-defined. The acoustic stack module 250 with the interposer 220 may be further assembled to a routing subassembly 241 by a z-axis interconnect layer 230. It is further appreciated that the piezo-electrically active layer (e.g. acoustic piezo layer 260) may comprise any of the following technologies, including PZT, LiNbO₃, Single-Crystal PIN-PMN-PT or PMN-PT materials, lead free piezoelectric materials, micro-machined pMUT or cMUT arrays, and 1-3 and 2-2 composites.

In one example embodiment, the Z-Axis interconnect layer 230 comprises Anisotropic Conductive Epoxy, or Anisotropic Conductive Film (ACF). The Z-Axis interconnect layer 230 may also be created using gold stud bumps on the surface of the routing subassembly(ies). In an example embodiment, the Z-Axis interconnect layer 230 is in the x-y plane, or azimuth-elevation plane. The Z-Axis interconnect layer 230 may comprise copper pillars which are grown through semiconductor processing on the routing assembly (e.g., on each routing subassembly (e.g. 241). The interposer 220 may be secured to the surface of the routing assembly with a nonconducting underfill epoxy, or the Z-Axis interconnect layer 230 may comprise printed or dispensed drops of Isotropic Conducting Adhesive. The interposer 220 may also be bonded to the surface of the routing assembly using a gold-to-gold thermos-compression bond. In an example embodiment, the routing subassembly 241 is a high-density routing substrate.

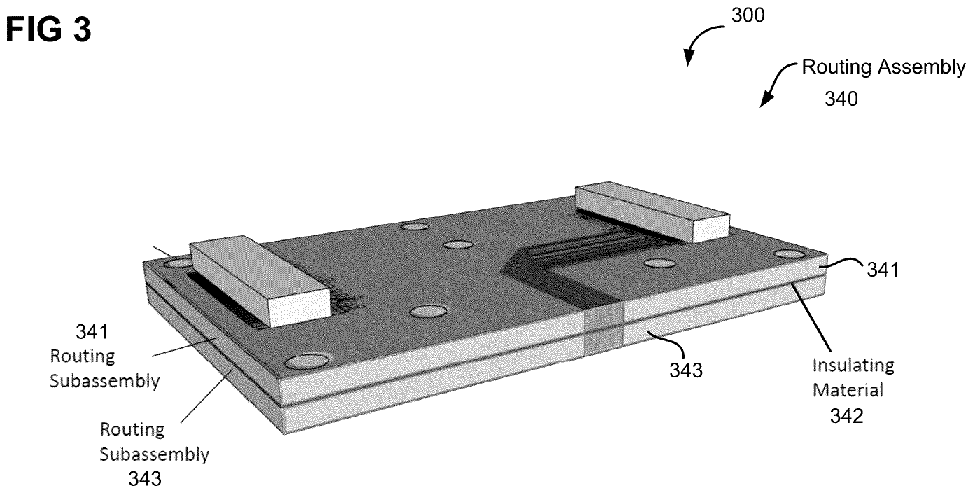

As is illustrated in FIG. 3, a routing assembly 340 may comprise multiple routing subassemblies (e.g., a first routing subassembly 341 and a second routing subassembly 343), each with a stack-up of two or more metallic routing layers (not shown). The routing subassemblies 341, 343 themselves may be preferably constructed using a commercially available organic build-up or High-Density Interconnect (HDI) Printed Circuit Board (PCB) fabrication process. It may be a fully rigid PCB or a flexible (FLEX) circuit or a circuit with rigid and flexible sections (RIGID-FLEX). It may further be constructed using High or Low Temperature Cofired Multilayer Ceramics (HTCC, or LTCC), or using a glass substrate. In addition, a semiconductor CMOS, SOI, or BCDMOS Back End of Line (BEOL) process could be used. In each of the above provided cases, the routing subassembly (341, 343) may comprise a routing substrate. Each routing substrate may further comprise multiple intervening copper layers between routing layers for electrical shielding to reduce crosstalk. In an example embodiment, an insulating material, adhesive, or insulating layer 342 may be located between the routing subassembly 341 and the routing subassembly 343.

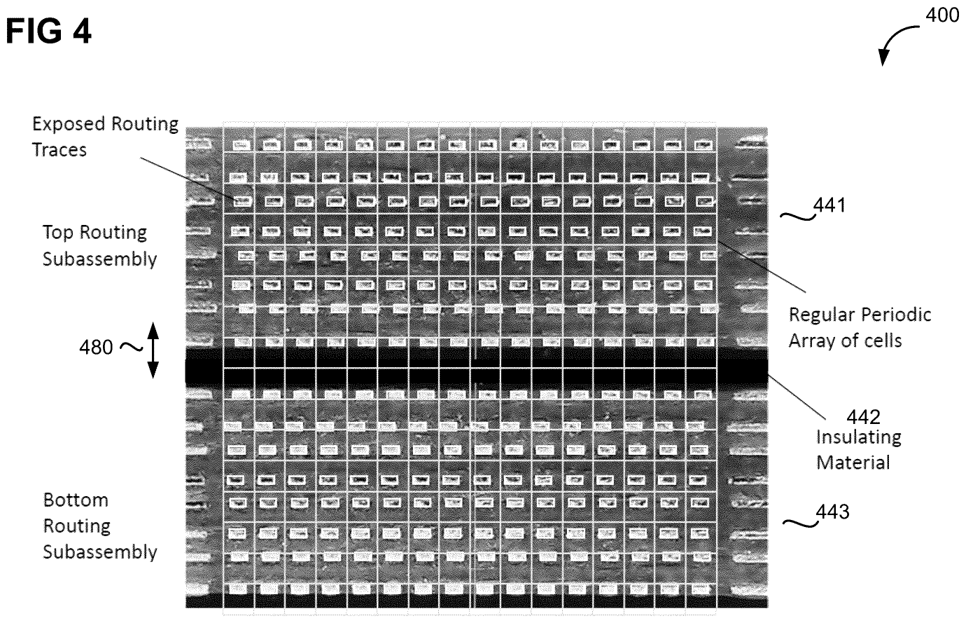

As further illustrated in FIG. 4, in an example embodiment, each pair of routing subassemblies (e.g. 441, 443) is separated by a layer of intervening insulating material 442 with thickness adapted so as to maintain periodicity of the plane-to-plane distance across the gap 480 between routing subassemblies. In particular, the periodicity of the routing planes of the routing substrate may be made to match the periodicity of the interposer routing channels in the dimension parallel to the principal routing plane dimension of the routing substrate. For fabrication processes in which the plane-to-plane pitch is smaller than the in-plane trace-to-trace pitch, it can be advantageous to orient the routing plane layers parallel to the azimuthal dimension to reduce pitch in that direction.

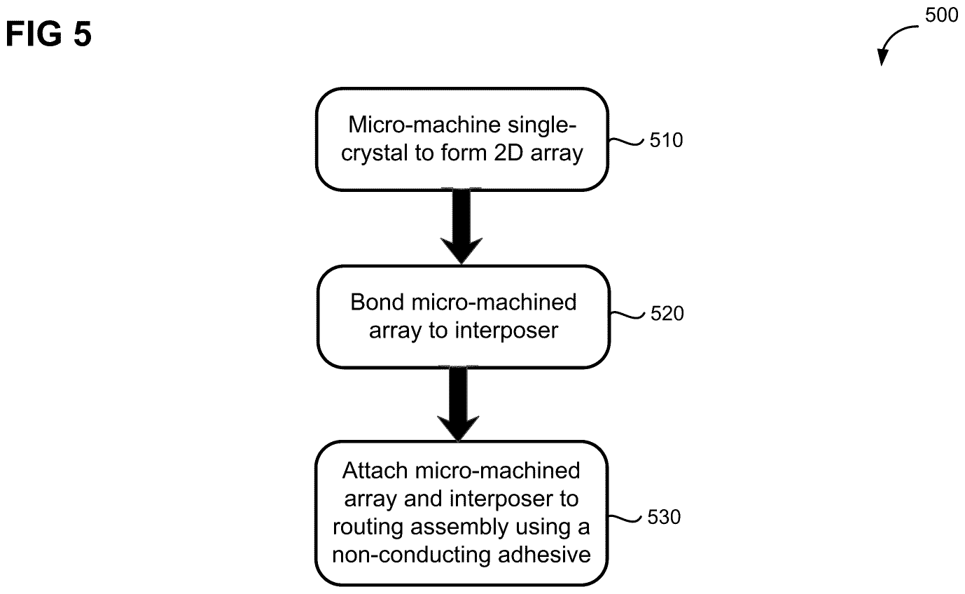

In accordance with an example embodiment, and as illustrated in FIG. 5, the structure of FIG. 1 may be accomplished using the following process 500: micro-machining a piezo-electric single-crystal material 260 to form a 2D array of elements (step 510), bonding the micro-machined array to an interposer (step 520), and finally attaching the micro-machined array and interposer to a routing assembly 340 using a non-conducting adhesive (step 530).

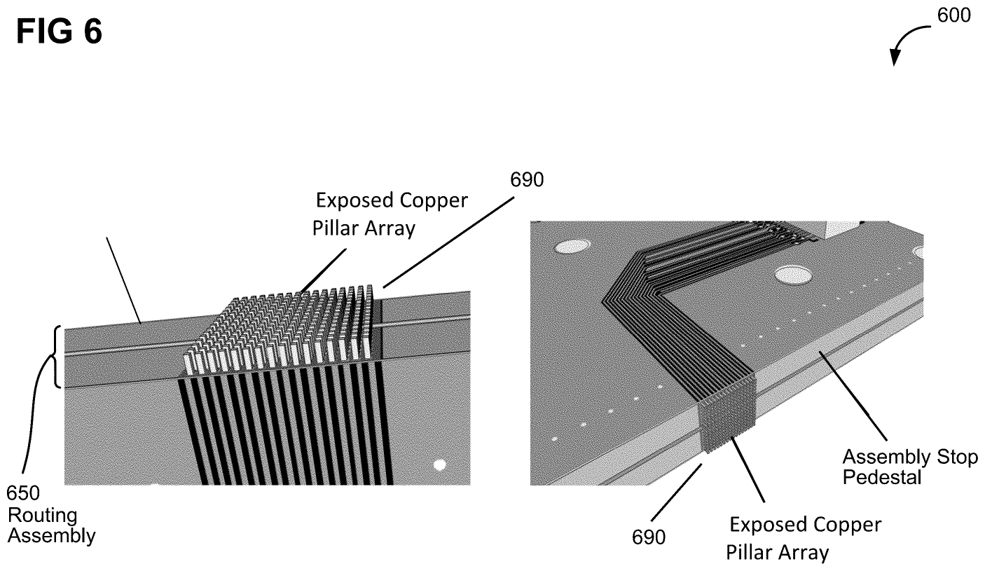

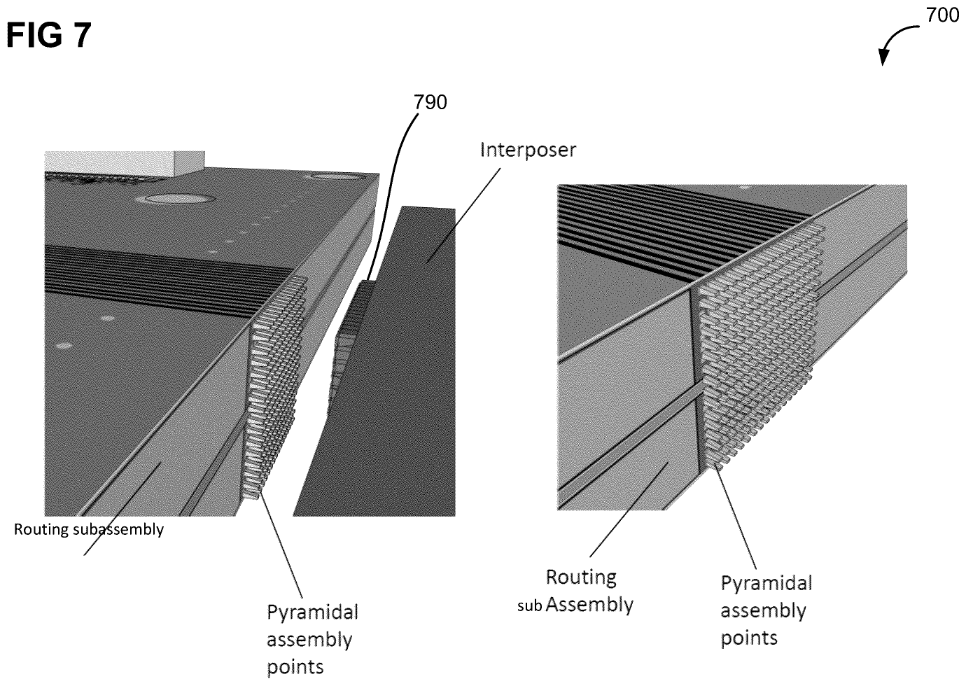

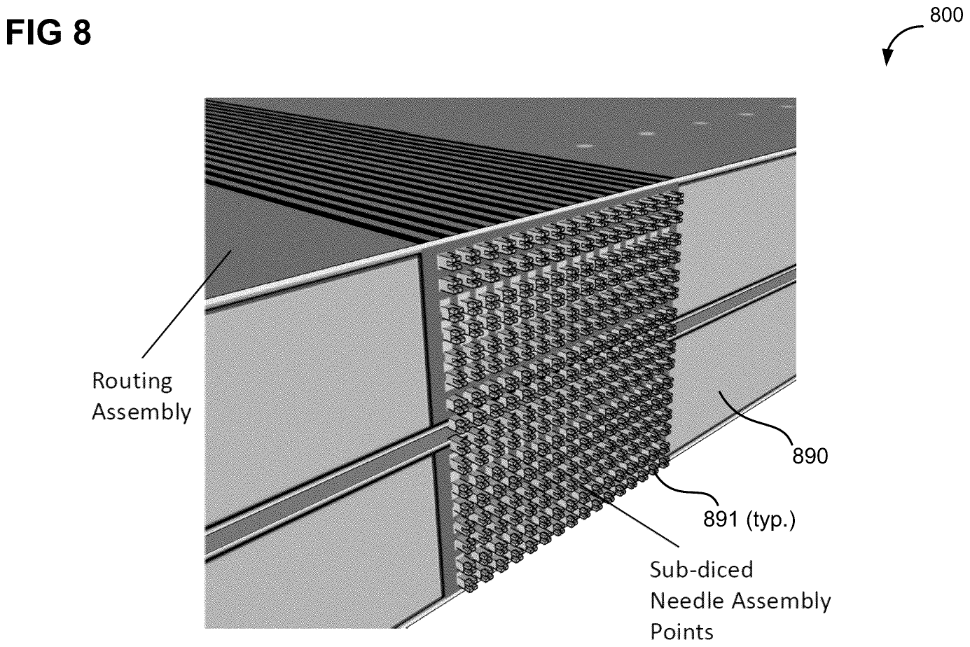

In accordance with an example embodiment, and as illustrated in FIG. 6, the routing assembly 650 has one edge perpendicular to its routing plane that comprises an array of exposed connection points 690, where each of the exposed connection points 690 corresponds to an endpoint of one routing trace embedded in a routing subassembly of the routing assembly 650. In one example embodiment, each of the exposed connection points 690 is free-standing from the routing subassembly in the shape of an exposed conducting pillar. In a further embodiment illustrated in FIG. 7, each exposed connection point is free-standing from the routing subassembly in the shape of an exposed conducting pyramid. The exposed connection points 690 further comprise an atmospherically inert layer of deposited metal to aid in producing ohmic electrical contacts to corresponding interposer connection points (790 in FIG. 7). As illustrated in FIG. 8, each of the exposed conducting pillars 890 may further be sub-diced to create multiple needles 891 that comprise an insertion assembly.

In a further example embodiment, the routing assembly 650 may be 3D-printed using a commercially available process that combines both non-conducting resin and conducting material to create arbitrary geometries of conducting wires held in place by non-conducting material. In this case, the free-standing connection points 690 integrated into the routing subassembly are incorporated into the 3D-printed design. The free-standing connection points 690 may be free-standing pillars and or pyramidal shape. In an example embodiment, the routing assembly 650 may further comprise assembly stops (not shown in FIG. 6) and the assembly stops may also be incorporated as part of the 3D-printed design. In addition, the 3D-printed routing assembly 650 may further comprise a frame (not shown in FIG. 6) which surrounds and supports the sensor array 110, and interposer 120.

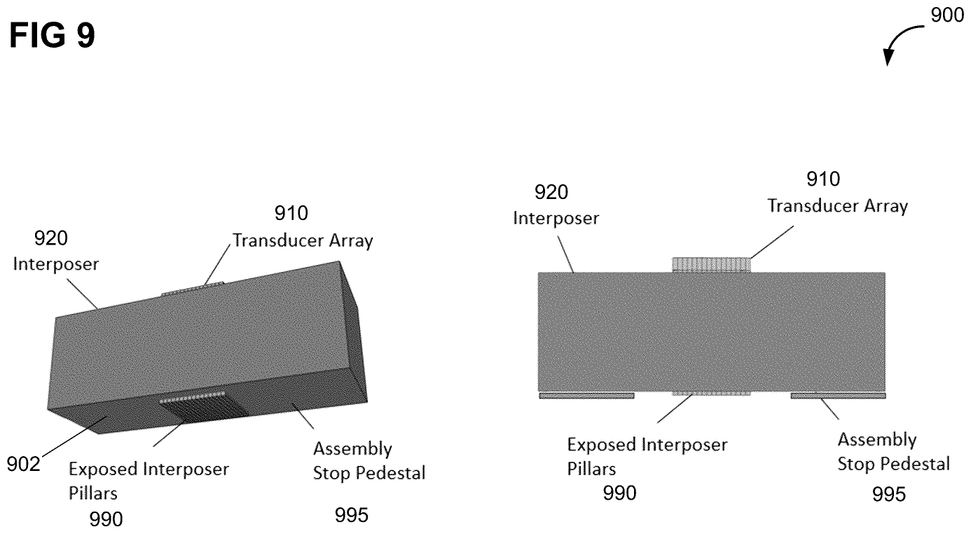

In accordance with an example embodiment, and as illustrated in FIG. 9, the side 902 of the interposer 920 opposite the transducer array 910 may comprise exposed connection points 990 that may be free-standing from the interposer 920. These exposed connection points 990 can take the form of free-standing pillars or pyramidal shaped columns. The surface of the exposed connection points 990 may be further processed to comprise an atmospherically inert layer of conducting deposited metal to facilitate ohmic electrical assembly. Additionally, the side 902 of the interposer 920 opposite to the transducer array 910 may comprise deep grooves that match pyramidal connection points on the edge surface of the routing substrate.

In another example embodiment as illustrated in FIG. 9, the bottom surface 902 of the interposer 920 further comprises an assembly stop support layer 995. This assembly stop support layer 995 may be integrated directly into the 3D-printed structure of the interposer 920 itself, or it may be created separately and bonded to the bottom surface 902 of the interposer 920 as a frame that surrounds the exposed interposer pillars. The purpose of the assembly stop support layer 995 is twofold: the first goal of the assembly stop support layer 995 is to provide a precisely defined depth of collapse for the assembly connection points 990 on either the interposer 920 side or the routing assembly side or both. The second purpose of the assembly stop support layer 995 is to provide a large surface area for adhesion of the interposer 920 and associated acoustic stack to the routing assembly. This large surface area is advantageous for improving robustness of the combined acoustic stack to routing assembly by providing ample area for the adhesive layer to grip the two sides of the assembly.

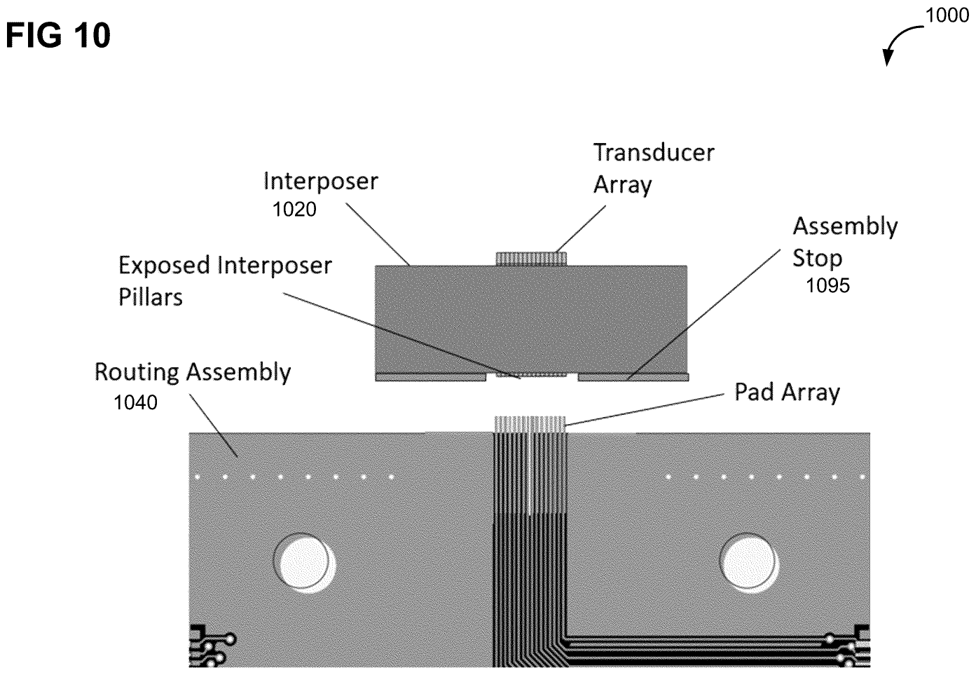

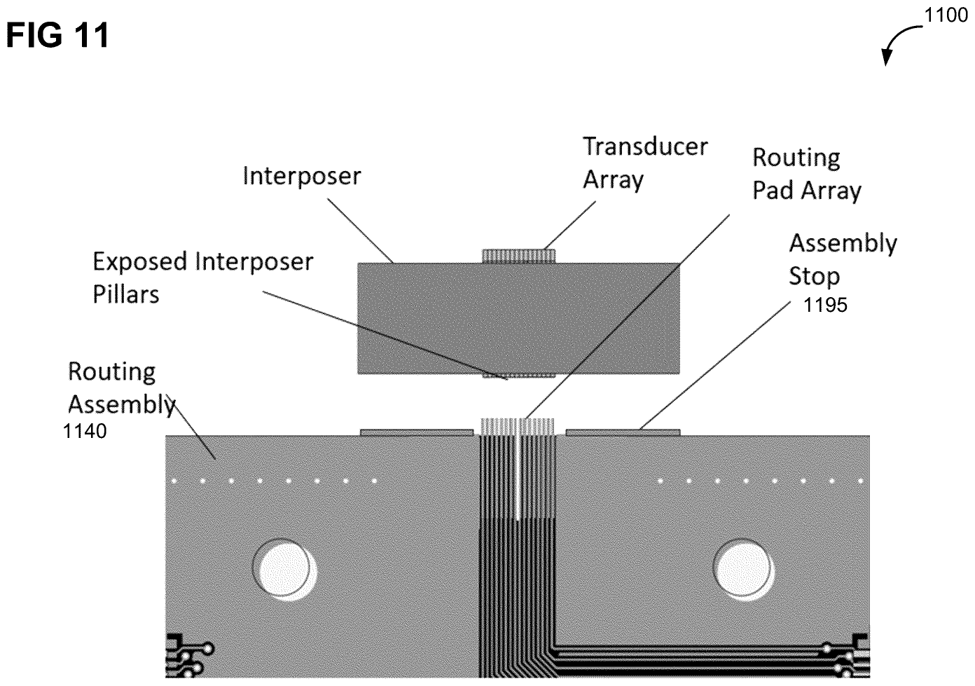

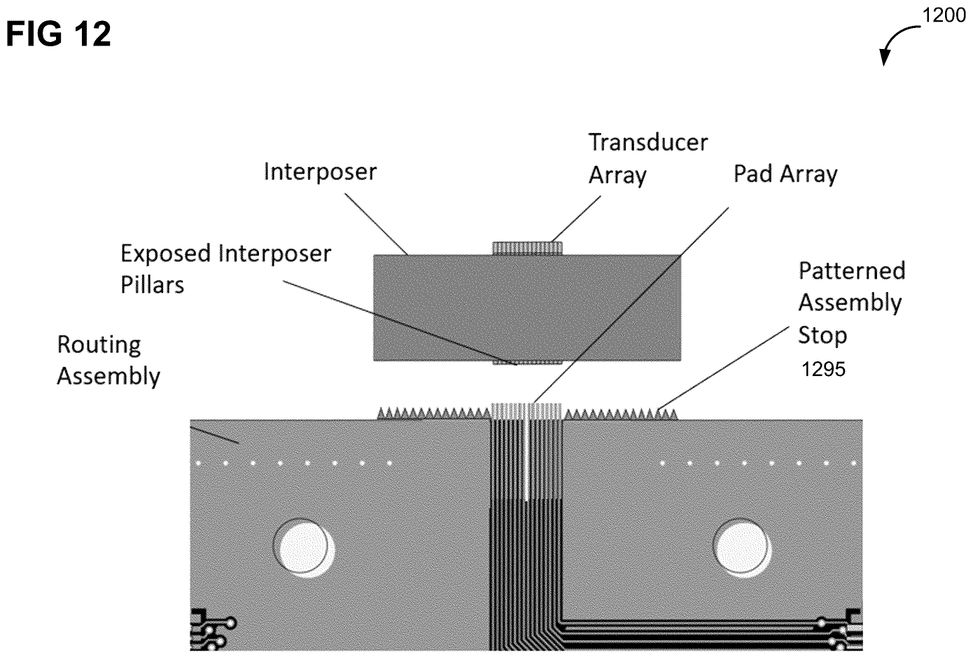

As illustrated in FIG. 10, in an example embodiment, the assembly stop support layer 1095 may comprise a 3D-printed frame of material with high modulus of elasticity that is bonded to both the interposer 1020 and the routing assembly 1040. The assembly stop support layer 1195 may also be contiguous with the routing assembly 1140 and formed out of the same material as the routing assembly 1140 as illustrated in FIG. 11. As further illustrated in FIG. 12, the assembly stop support layer 1295 may also be patterned for increased surface area of bonding.

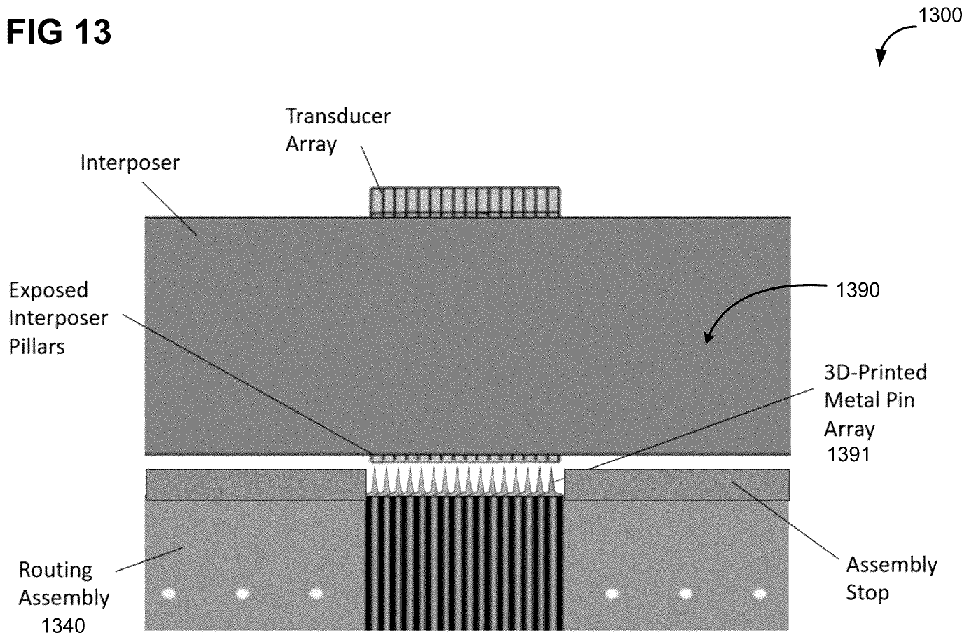

In an example embodiment, and as illustrated in FIG. 13, the assembly connection points 1390 in the modular acoustic sensor assembly 1300 comprise a 3D-printed metal grid of pins 1391. The pins 1391 may form a regular matrix in two dimensions and are connected to each other by metal links that are incorporated into the 3D-printed design. In a further example embodiment, the 3D-printed metal grid of pins 1391 is attached to the routing assembly 1340 by a metallic soldering process.

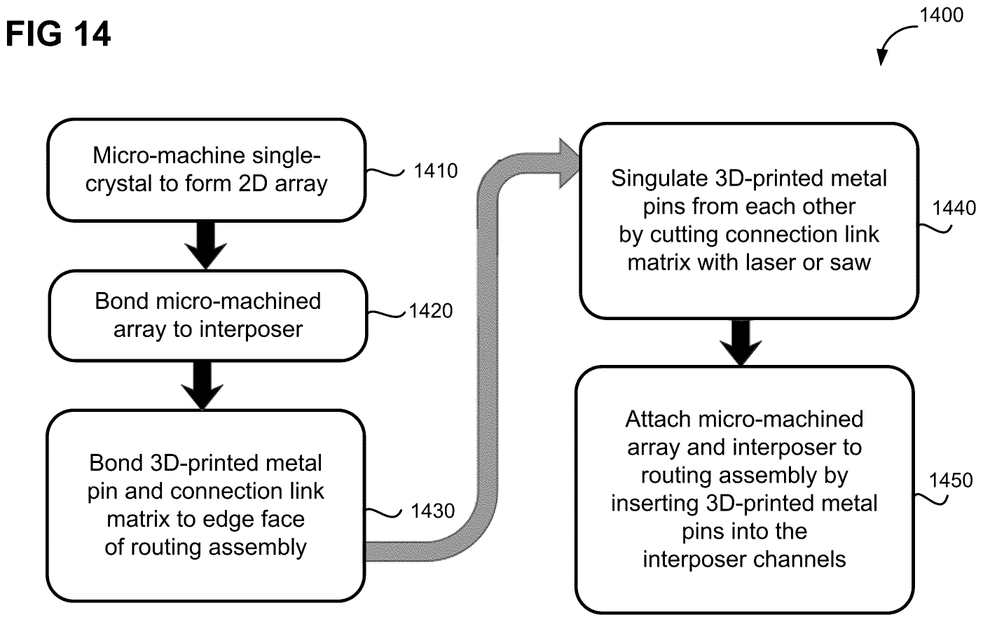

In an example embodiment, and as illustrated in FIG. 14, the modular acoustic sensor assembly 1300 of FIG. 13 may be accomplished using the following process 1400: micro-machining a piezo-electric single-crystal material to form a 2D array of elements (step 1410), bonding the micro-machined array to an interposer (step 1420), bonding a 3D-printed metal pin and connection link matrix to the edge face of a routing assembly using a thermo-compression or solder reflow process (step 1430), singulating the 3D-printed metal pins from each other by cutting the connection link matrix using a dicing saw or a laser cutting process (step 1440), attaching the micro-machined array and interposer to the routing assembly by inserting the 3D-printed metal pins into the interposer channel (step 1450).

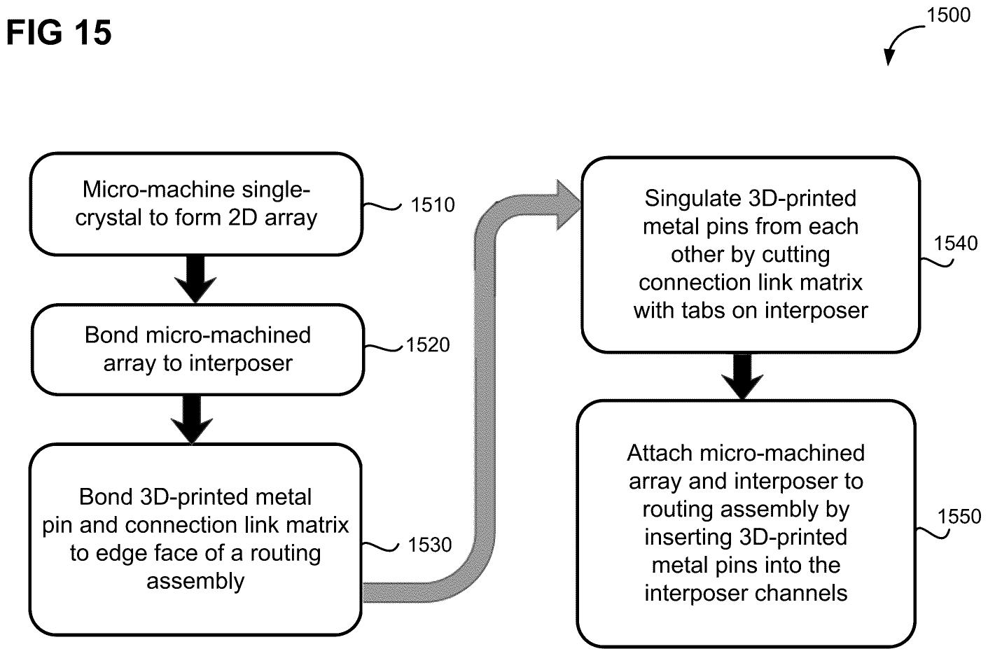

In another example embodiment, and as illustrated in FIG. 15, the modular acoustic sensor assembly 1300 of FIG. 13 may be accomplished using the following process 1500: micro-machining a piezo-electric single-crystal material 260 to form a 2D array of elements (step 1510), bonding the micro-machined array to an interposer (step 1520), bonding a 3D-printed metal pin and connection link matrix to the edge face of a routing substrate using a thermo-compression or solder reflow process (step 1530), singulating the 3D-printed metal pins from each other by cutting the connection link matrix as part of the press-fit assembly process using protruding tabs on the bottom surface of the mating interposer (step 1540), and attaching the micro-machined array and interposer to the routing assembly by inserting the 3D-printed metal pins into the interposer channel (step 1550).

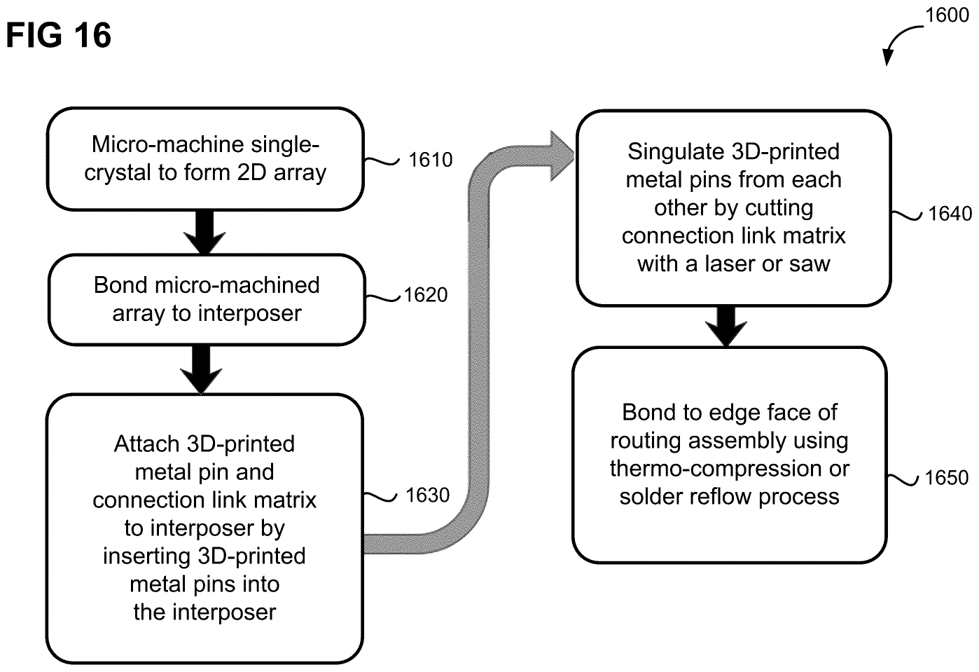

In another example embodiment, and as illustrated in FIG. 16, the modular acoustic sensor assembly 1300 of FIG. 13 may be accomplished using the following process 1600: micro-machining a piezo-electric single-crystal material 260 to form a 2D array of elements (step 1610), bonding the micro-machined array to an interposer (step 1620), attaching a 3D-printed metal pin and connection link matrix to the micro-machined array and interposer by inserting the 3D-printed metal pins into the interposer channel material (step 1630), singulating the 3D-printed metal pins from each other by cutting the connection link matrix using a dicing saw or a laser cutting process (step 1640), and bonding the edge face of a routing assembly to the 3D-printed metal pins using a thermo-compression or solder reflow process (step 1650).

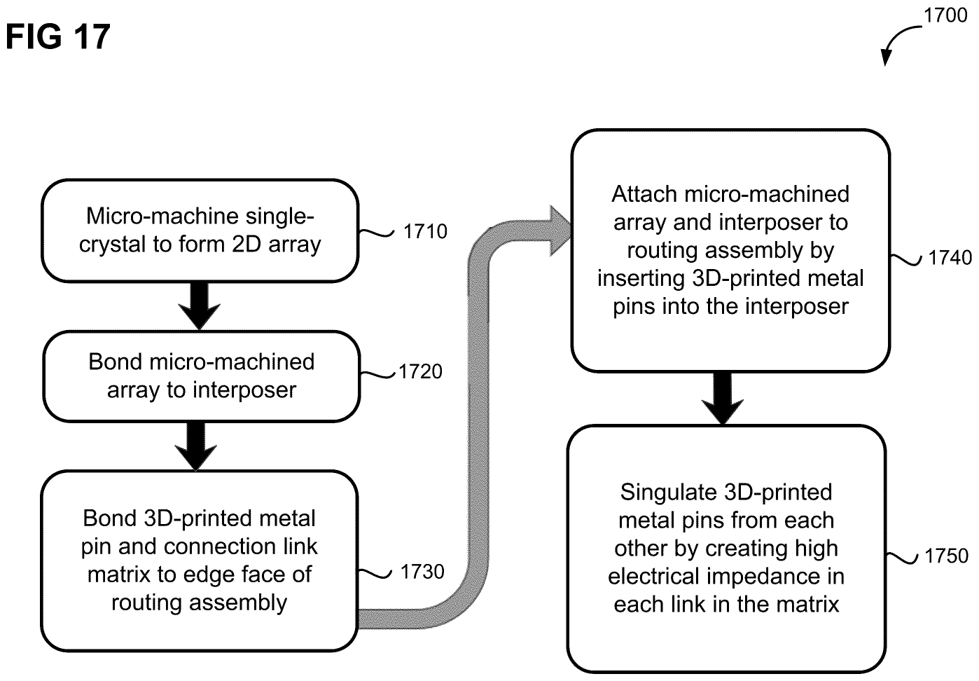

In another example embodiment, and as illustrated in FIG. 17, the modular acoustic sensor assembly 1300 of FIG. 13 may be accomplished using the following process 1700: micro-machining a piezo-electric single-crystal material 260 to form a 2D array of elements (step 1710), bonding the micro-machined array to an interposer (step 1720), bonding a 3D-printed metal pin and connection link matrix to the edge face of a routing assembly using a thermo-compression or solder reflow process (step 1730), attaching the micro-machined array and interposer to the routing assembly by inserting the 3D-printed metal pins into the interposer channel (step 1740), and singulating the 3D-printed metal pins from each other by creating high electrical impedance in each link in the matrix (step 1750). The high electrical impedance can be created by a fabrication test system by individually activating pairs of element channels at high voltage to ground, thereby effectively burning the link using a fusing action as is commonly used for programming microchips in use.

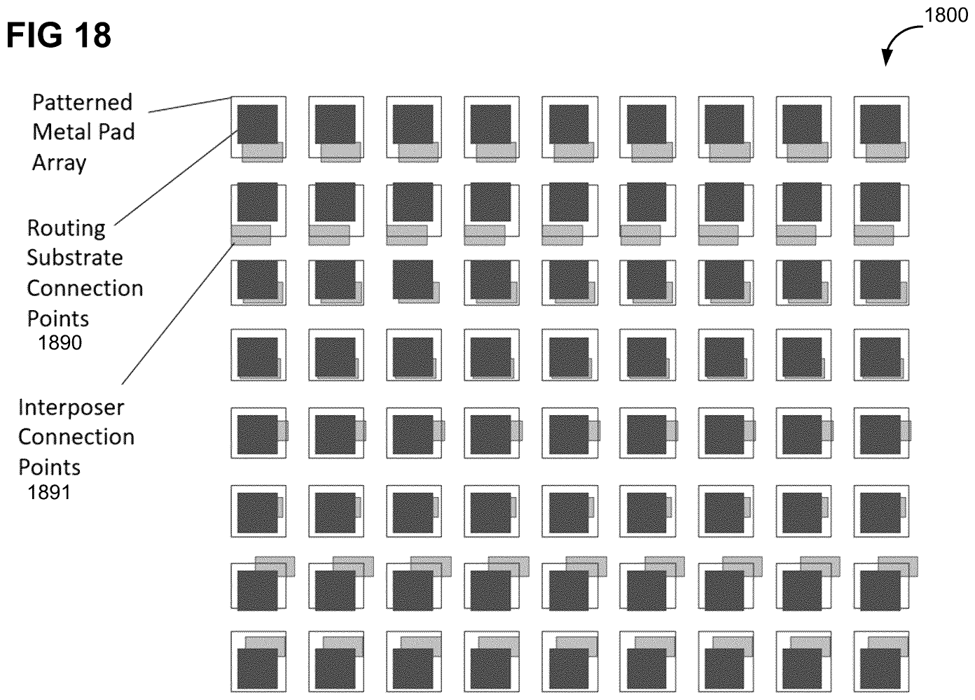

As illustrated in FIG. 18, in an example embodiment, the pitch of the interposer connection points 1891 in one or two dimensions may differ randomly from the pitch of the interconnection points 1890 of the routing subassembly. In this case, an intervening metal pad array may be applied to the interposer connection points 1891 and patterned so as to reroute signals to match the interconnection points 1890 on the routing subassembly. In a further embodiment, a 3D-printed metal grid of pins may be applied to the interposer connection points 1891 and patterned so as to reroute signals to match the interconnection points 1890 on the routing subassembly. Automated optical inspection and deep learning algorithms may be applied to tailor the metal pad array or 3D-printed metal grid of pins to accomplish the re-routing of signals.

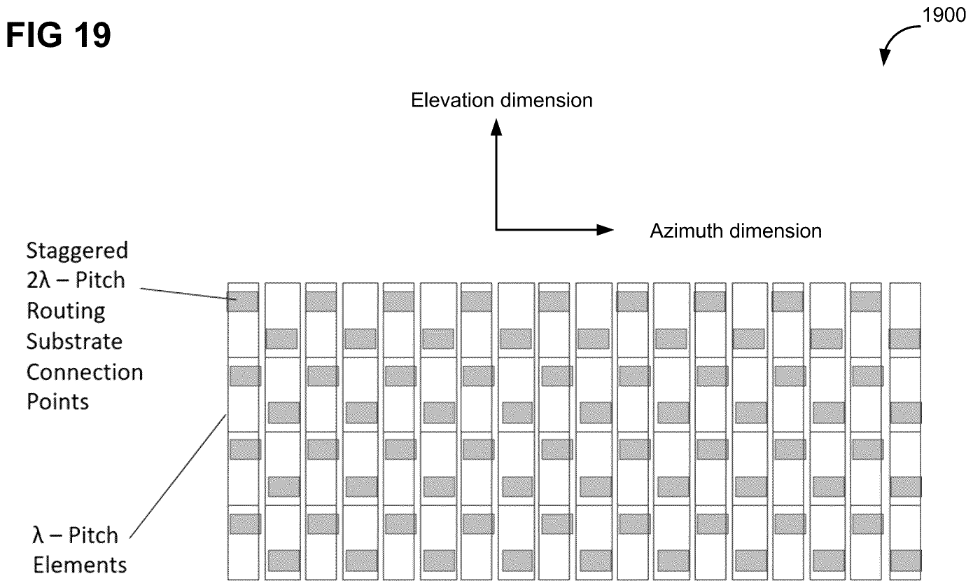

In some routing substrate fabrication processes, it may be possible for vendors to implement staggered traces laterally and in this case, it is advantageous to distribute the azimuthal dimension of the array along the staggering direction as a way to realize half pitch elements. In view of this, in a further example embodiment illustrated in FIG. 19, the routing substrate comprises half-pitch assembly pads implemented by staggered distribution of alternating routing layers. This distribution of pads is advantageous when implementing fine pitch 1.5D and 1.75D arrays that must have optimized resolution in the azimuthal dimension and can tolerate larger element size in the elevation dimension.

It is further appreciated that the routing substrate may itself contain active electronics in the form of ASICs that are assembled to its surface either by flip-chip assembly or chip-on-board wire-bonding or standard surface-mount reflow processes. These could be any combination of high voltage and low voltage circuitry for transmit and receive processing of ultrasound signals and may further comprise local digital or analog beam-forming signal capability. The arrays may be further configured to implement reduced channel beamforming architectures such as row-column and sparsely populated arrays and may further include local multiplexing and grouping of the element channels as well as signal buffering.

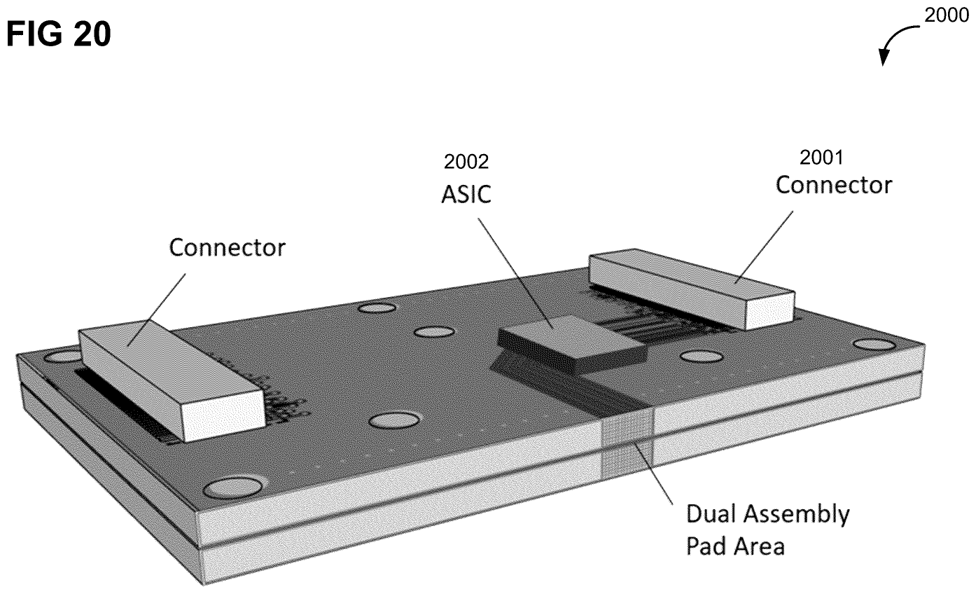

In a further embodiment illustrated in FIG. 1, the routing assembly 145 houses one or more electrical connectors 101 which can be used to provide signal, control and power supply connections to the imaging system. In addition, as illustrated in FIG. 20, the routing assembly may further house one or more interface ASICs 2002. The one or more respective electrical connectors 2001 in FIG. 20 may be wired to respective array elements in electrical contact with the respective routing subassembly. Additionally, the one or more respective ASICs 2002 may be wired to respective array elements in electrical contact with the respective routing subassembly. In a further embodiment, each routing subassembly houses one or more respective electrical connectors 2001 that are wired to respective ASICs 2002 that are wired to respective array elements in electrical contact with the respective routing subassembly. The ASICs 2002 functionally provide any combination of multiplexing, transmit-beamforming, receive-beamforming, buffering or electrical impedance matching.

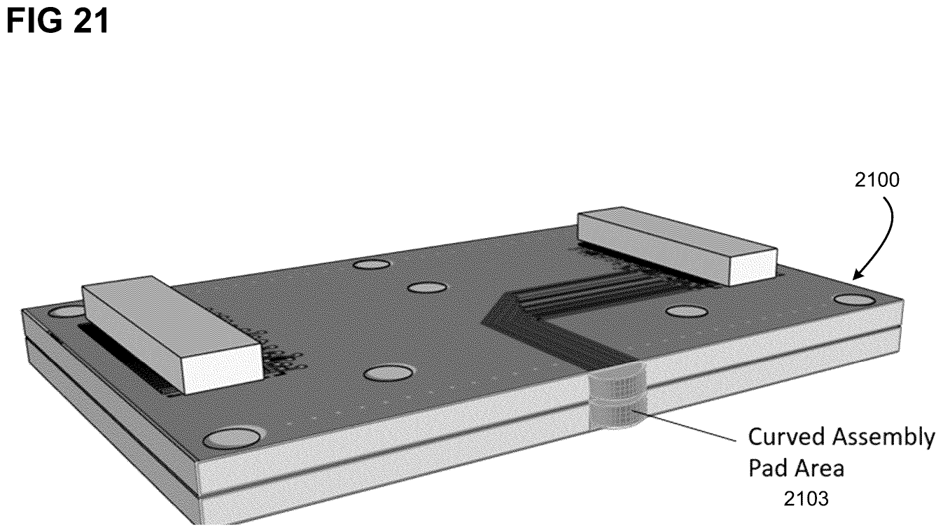

In a further embodiment illustrated in FIG. 21, the acoustic stack module 2100 has a curved shape 2103 to facilitate assembly of a transducer array that is curved in one or two dimensions. The use of both concave and convex curved arrays is advantageous for applications where a large field of view is covered using an array that operates in a standard linear mode in azimuth and as a 1.75D array in elevation. In an example embodiment, the acoustic stack module 2100 is manufactured with the curved shape 2103 and then attached to the routing assembly.

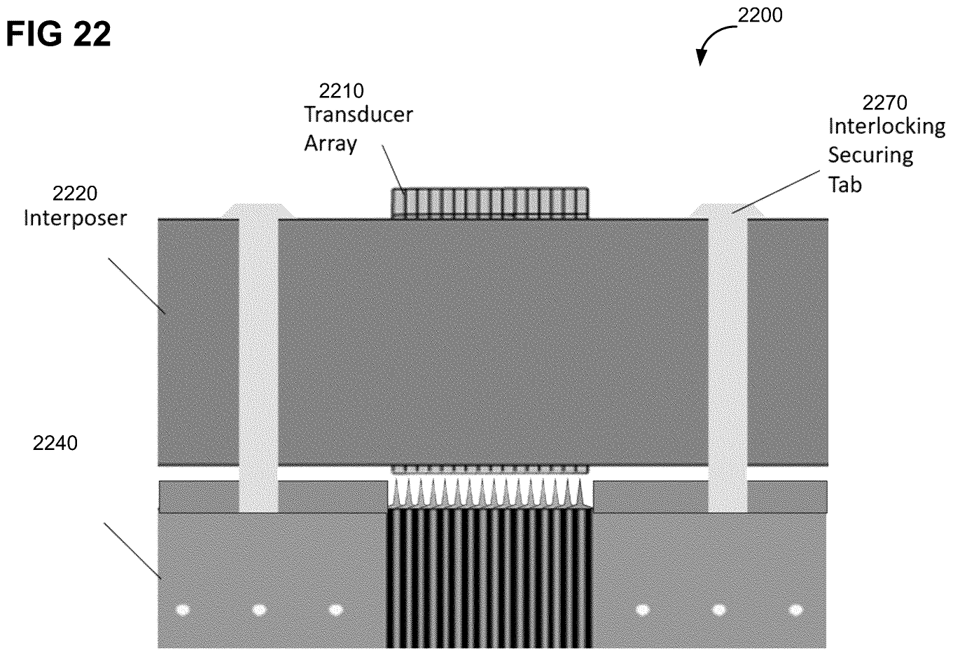

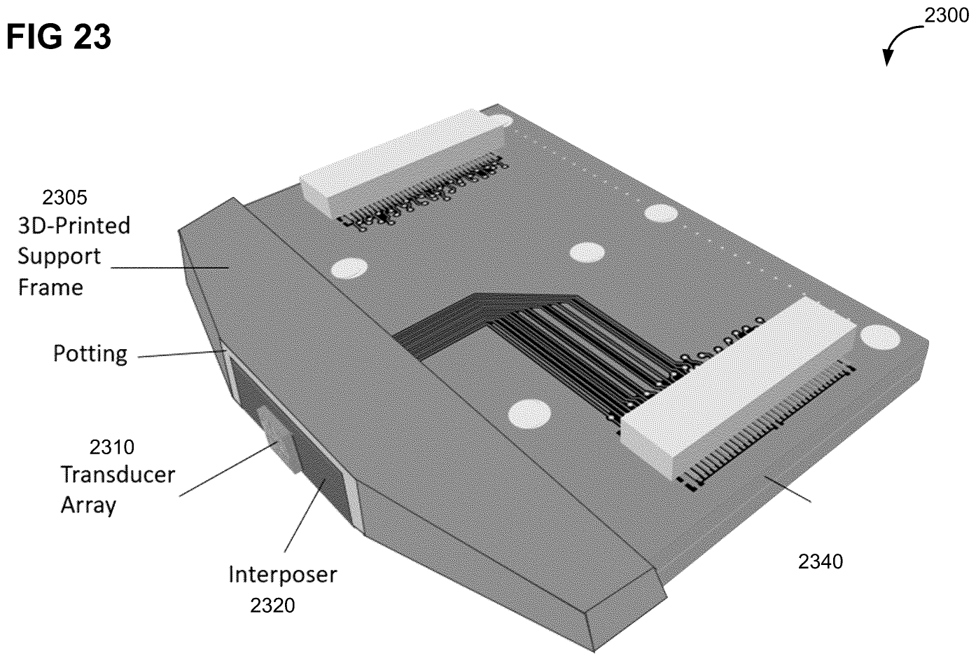

In a further embodiment illustrated in FIG. 22, the modular acoustic sensor assembly 2200 comprises a two-dimensional acoustic sensor array 2210, an electrically conducting and acoustically attenuating interposer 2220, a routing subassembly 2240, and interlocking tabs or screws 2270 that secures the interposer 2220 to the routing subassembly 2240, where at least one electrical routing plane of the interposer 2220 is substantially parallel to the principal electrical routing plane of the routing subassembly 2240. In addition, as is illustrated in FIG. 23, a 3D printed frame 2305 is configured to surround and support the sensor array 2310, interposer 2320, and routing subassembly 2340. In an example embodiment, the 3D printed frame 2305 may be used to secure these by potting with a non-conducting epoxy.

The arrays themselves may further implement full λ/2-pitch acoustic elements capable of 3D volume acquisition by phased-array steering in both azimuthal and elevation planes, or they may instead implement λ (or greater) pitch element arrays for 1.75D, 1.5D, or 1.25D type scanning linear arrays with improved elevational focusing and electronically translated scanning along the elevation dimensions to build up successive planes of the 3D volume. An important aspect of the present disclosure is the realization of large area arrays at very fine pitch, and this may advantageously be accomplished using an aggregate modular acoustic sensor comprising multiple modular acoustic sensor assemblies that are tiled next to each other in one or two dimensions.

In an example embodiment, a modular acoustic sensor assembly 100 may comprise a 2D array of transducer elements 110 with (for example) 32 elements in elevation and 64 elements in azimuth. The modular acoustic sensor assembly 100 may be formed by two routing subassemblies (e.g. 341 and 343), wherein each routing subassembly has 16 routing layers (a PCB having 16 routing layers). In this example embodiment, the two routing subassemblies are bonded to each other and connected to an acoustic stack module 250 comprising the interposer 220 and acoustic sensor array 110. Of course, any suitable number of elements and stacked routing subassemblies may be used.

### STATEMENTS

In various example embodiments, the acoustic sensor array comprises micro-machined single-crystal ultrasound transducer material 260.

In various example embodiments, the side of the interposer opposite the transducer array comprises exposed connection points that are free-standing from the interposer.

In various example embodiments, each exposed connection point comprises an atmospherically inert layer of deposited metal.

In various example embodiments, the routing substrate comprises any one of a PCB, HDI, Ceramic, Silicon, Glass, Rigid-Flex or fully Flex Circuit substrate.

In various example embodiments, the ASICs functionally provide any combination of multiplexing, transmit-beamforming, receive-beamforming, buffering or electrical impedance matching.

In various example embodiments, assembly stops are incorporated as part of the 3D-printed design.

In various example embodiments, a process for building a modular acoustic sensor assembly is disclosed comprising the steps of: micro-machining a piezo-electric single-crystal material to form a 2D array of elements; bonding the micro-machined array to an interposer; and attaching the micro-machined array and interposer to a routing subassembly using a non-conducting adhesive layer.

In various example embodiments, a process for building a modular acoustic sensor assembly is disclosed comprising the steps of: micro-machining a piezo-electric single-crystal material to form a 2D array of elements; bonding the micro-machined array to an interposer; attaching a 3D-printed metal pin and connection link matrix to the micro-machined array and interposer by inserting the 3D-printed metal pins into the interposer channel material; singulating the 3D-printed metal pins from each other by cutting the connection link matrix using a dicing saw or a laser cutting process; and bonding the edge face of a routing subassembly to the 3D-printed metal pins using a thermo-compression or solder reflow process.

In various example embodiments, a process for building a modular acoustic sensor assembly is disclosed comprising the steps of: micro-machining a piezo-electric single-crystal material to form a 2D array of elements; bonding the micro-machined array to an interposer; attaching a 3D-printed metal pin and connection link matrix to the micro-machined array and interposer by inserting the 3D-printed metal pins into the interposer channel material; bonding the edge face of a routing subassembly to the 3D-printed metal pins using a thermo-compression or solder reflow process; and singulating the 3D-printed metal pins from each other by creating high electrical impedance in each link in the matrix by high current fusing of neighbor channels.

In accordance with various example embodiments, the 3D-printed routing assembly further comprises a frame which surrounds and supports the sensor array, and interposer.

In accordance with various example embodiments, a modular acoustic sensor assembly for operation at high frequency is disclosed, comprising: a two-dimensional acoustic sensor array; an electrically conducting and acoustically attenuating interposer; a routing subassembly; and interlocking tabs or screw that secures the interposer to the routing subassembly; wherein at least one electrical routing plane of the interposer is substantially parallel to the principal electrical routing plane of the routing subassembly.

In an example embodiment, the pitch of the interposer connection points in one or two dimensions differs randomly from the pitch of the interconnection points of the routing subassembly.

In an example embodiment, an intervening metal pad array is applied to the interposer connection points and patterned so as to reroute signals to match the interconnection points on the routing subassembly.

In an example embodiment, a 3D-printed metal grid of pins is applied to the interposer connection points and patterned so as to reroute signals to match the interconnection points on the routing subassembly.

In an example embodiment, rerouting of the signals to match the interconnection points on the routing subassembly is accomplished by expansion of the interposer pitch from top to bottom of the interposer in one or two dimensions.

In an example embodiment, the routing subassembly further comprises multiple intervening copper layers between routing layers for electrical shielding to reduce crosstalk.

In an example embodiment, each exposed conducting pillar is sub-diced to create multiple needles to facilitate insertion assembly.

In an example embodiment, the assembly further comprises a 3D printed frame which surrounds and supports the sensor array, the interposer and the routing assembly and is secured by potting with a non-conducting epoxy.

In accordance with various example embodiments, an aggregate modular acoustic sensor is disclosed comprising multiple modular acoustic sensor assemblies that are tiled next to each other in one or two dimensions.

In an example embodiment, a routing substrate may comprise a Printed Circuit Board (PCB), a flex circuit, an LTCC ceramic substrate or the like.

What is claimed is:

1. A modular acoustic sensor assembly for operation at high frequency comprising:
a two-dimensional acoustic sensor array;
an electrically conducting and acoustically attenuating interposer;
an assembly connection layer; and
a first routing subassembly, wherein at least one electrical routing plane of the interposer is substantially parallel to a principal electrical routing plane of the first routing subassembly.

2. The modular acoustic sensor assembly of claim 1, wherein the interposer comprises a 3D printed grid frame.

3. The modular acoustic sensor assembly of claim 1, wherein a periodicity of routing planes of the first routing subassembly matches the periodicity of routing channels of the interposer in the dimension parallel to a principal routing plane dimension of the first routing subassembly.

4. The modular acoustic sensor assembly of claim 1, wherein a side of the interposer opposite to the acoustic sensor array comprises deep grooves that match pyramidal connection points on an edge surface of the first routing subassembly.

5. The modular acoustic sensor assembly of claim 1, wherein the first routing subassembly comprises half-pitch assembly pads implemented by staggered distribution of alternating routing layers.

6. The modular acoustic sensor assembly of claim 1, further comprising a second routing subassembly, wherein the first routing subassembly and the second routing subassembly are separated by a layer of intervening insulating material with thickness adapted so as to maintain periodicity of the plane-to-plane distance across a gap between the first and second routing subassemblies, and wherein a routing assembly comprises the first routing subassembly and the second routing subassembly.

7. The modular acoustic sensor assembly of claim 1, wherein the assembly connection layer comprises one of:
a non-conducting adhesive;
an anisotropic conductive adhesive;
an anisotropic conductive film; or
a 3D-printed metal grid of pins.

8. The modular acoustic sensor assembly of claim 7, wherein the assembly connection layer comprises the 3D-printed metal grid of pins, and wherein one of:
the 3D-printed metal grid of pins is attached to the first routing subassembly by a metallic soldering process; and
the 3D-printed metal grid of pins form a regular matrix in two dimensions and are connected to each other by metal links that are incorporated into the 3D-printed design.

9. The modular acoustic sensor assembly of claim 8, comprising multiple routing subassemblies, wherein one of:
each routing subassembly houses one or more respective electrical connectors that are wired to respective array elements in electrical contact with the respective routing subassembly;
each routing subassembly houses one or more respective ASICs that are wired to respective array elements in electrical contact with the respective routing subassembly; or
each routing subassembly houses one or more respective electrical connectors that are wired to respective ASICs that are wired to respective array elements in electrical contact with the respective routing subassembly.

10. The modular acoustic sensor assembly of claim 8, wherein an acoustic stack module comprises the acoustic sensor array, the interposer, and the assembly connection layer, wherein the acoustic stack module has a curved shape to facilitate assembly of transducer arrays that are curved in one or two dimensions.

11. The modular acoustic sensor assembly of claim 8, wherein one edge perpendicular to a routing plane of the first routing subassembly comprises an array of exposed connection points, where each of the exposed connection points corresponds to an endpoint of one routing trace embedded in the first routing subassembly.

12. The modular acoustic sensor assembly of claim 11, wherein one of:
each exposed connection point is free-standing from the first routing subassembly in the shape of an exposed conducting pillar;
each exposed connection point is free-standing from the first routing subassembly in the shape of an exposed conducting pyramid; or
each exposed connection point comprises an atmospherically inert layer of deposited metal.

13. The modular acoustic sensor assembly of claim 1, further comprising an assembly stop support layer, wherein the assembly stop support layer comprises a frame of material with high modulus of elasticity that is bonded to both the interposer and the first routing subassembly, and wherein the assembly stop support layer is one of:
contiguous with the first routing subassembly and formed out of the same material as the first routing subassembly; or
contiguous with the interposer and formed out of the same material as the interposer.

14. The modular acoustic sensor assembly of claim 13, wherein assembly stops are patterned for increased surface area of bonding.

15. The modular acoustic sensor assembly of claim 1, wherein the first routing subassembly is 3D-printed.

16. The modular acoustic sensor assembly claim 15, wherein free-standing connection points are incorporated into the 3D-printed first routing subassembly.

17. The modular acoustic sensor assembly of claim 16, wherein one of:
the free-standing connection points are free-standing pillars;
the free-standing connection points are free-standing pyramids; or
assembly stops are incorporated as part of the 3D-printed first routing subassembly.

18. The modular acoustic sensor assembly of claim 1, wherein a side of the interposer opposite the sensor array comprises exposed connection points that are free-standing from the interposer.

19. A process for building a modular acoustic sensor assembly comprising the steps of:
micro-machining a piezo-electric single-crystal material to form a 2D array of elements;
bonding the micro-machined array to an interposer; and one of:
bonding a 3D-printed metal pin and connection link matrix to an edge face of a first routing subassembly using a thermo-compression or solder reflow process;
singulating the 3D-printed metal pins from each other by cutting the connection link matrix using a dicing saw or a laser cutting process; and
attaching the micro-machined array and the interposer the first routing subassembly by inserting the 3D-printed metal pins into channel material of the interposer; or
attaching a 3D-printed metal pin and connection link matrix to the micro-machined array and interposer by inserting the 3D-printed metal pins into channel material of the interposer;
singulating the 3D-printed metal pins from each other by cutting the connection link matrix using a dicing saw or a laser cutting process; and
bonding the edge face of the first routing subassembly to the 3D-printed metal pins using the thermo-compression or solder reflow process.

20. A process for building a modular acoustic sensor assembly comprising the steps of:
micro-machining a piezo-electric single-crystal material to form a 2D array of elements;
bonding the micro-machined array to an interposer;
bonding a 3D-printed metal pin and connection link matrix to an edge face of a first routing subassembly using a thermo-compression or solder reflow process;
singulating the 3D-printed metal pins from each other by cutting the connection link matrix as part of a press-fit assembly process using protruding tabs on a bottom surface of a mating interposer; and
attaching the micro-machined array and the interposer to the first routing subassembly by inserting the 3D-printed metal pins into channel material of the interposer.
